(19) United States
(12) Patent Application Publication
(10) Pub. No.: US 2025/0212914 A1
(43) Pub. Date: Jul. 3, 2025

(54) OXIDATION STABILISED BIOMASS MATERIAL AND PROCESS

(71) Applicant: Unibio A/S, Roskilde (DK)

(72) Inventors: Theis Sommer, Roskilde (DK); Ida-Maria Heberg Thylkjær, Roskilde (DK); Michael Elleskov, Roskilde (DK); Michael Jensen, Roskilde (DK)

(21) Appl. No.: 18/875,532

(22) PCT Filed: Jun. 15, 2023
(86) PCT No.: PCT/EP2023/066039

§ 371 Date: Dec. 16, 2024

(30) Foreign Application Priority Data
Jun. 17, 2022 (EP) 22179681.6

(51) Int. Cl. A23K 50/80; A23K 10/12; A23K 20/105; A23K 20/147; A23K 40/10

(52) U.S. Cl. CPC A23K 50/80; A23K 10/12; A23K 20/105; A23K 20/147; A23K 40/10

(57) ABSTRACT

A process is provided for producing a biomass material which is stabilised against oxidation. In particular, the process comprises the steps of: treating the concentrated biomass material with an antioxidant; and adjusting the pH of the concentrated biomass material to a pH of 7.0 or below. By means of these steps, a synergistic stabilisation of the biomass material is seen. An oxidation-stabilised biomass material is also provided, as well as an aquatic feed product, comprising the oxidation-stabilised biomass material.

### TECHNICAL FIELD

A process is provided for producing a biomass material which is stabilised against oxidation. In particular, the process comprises the steps of: treating the concentrated biomass material with an antioxidant; and adjusting the pH of the concentrated biomass material to a pH of 7.0 or below. By means of these steps, a synergistic stabilisation of the biomass material is seen. An oxidation-stabilised biomass material is also provided, as well as an aquatic feed product, comprising the oxidation-stabilised biomass material.

### BACKGROUND

The methanotrophic bacterium Methylococcus capsulatus is a non-commensal bacterium found ubiquitously in nature. It metabolizes methane, e.g., from natural gas, into biomass, CO₂ and water. Being rich in protein, M. capsulatus can be used as a protein supplement in animal feed and is also of interest for human consumption. The fermentation of this bacterium as a protein source for both animal and human consumption may contribute to satisfying the world's need for dietary protein in a way which is more environmentally friendly than conventional protein production industries.

Normally, biomass material includes fats and transition metals. Transition metals are present in biomass material as a result of the fermentation medium used in production of the biomass material. Attempts have been made to sequester transition metals, e.g. by including one or more chelating agents, but this has not proven to reduce oxidation.

Both fats and transition metals can both contribute to the production of oxidation products that can—in turn—lower the quality of the product as well as its storage lifetime. Furthermore, biomass product is typically heat-treated during production (e.g. spray-dried and pelletised) which are also believed to initiate or accelerate oxidation. Additional factors which are believed to accelerate oxidation include accessibility to oxygen and light, and the water content of the biomass product.

A particular problem with fish feed arises, as fish will not eat oxidised feed. One object of the invention to provide processes and biomass products with improved palatability.

### SUMMARY

It has been found by the present inventor(s) that, by adding antioxidant and changing or controlling the pH, it is possible to protect the biomass product against oxidation. The pH regulator and antioxidant act in synergy to provide an increase in stability which is not evident when using these components separately. In other words, treatment with antioxidant alone, acidity regulator alone, or acidity regulator to the incorrect pH does not show an increase in oxidation stability.

Therefore, in a first aspect the present invention relates to a process for producing a biomass material which is stabilised against oxidation, said process comprising the steps of:
a. fermenting at least one methanotroph in a fermentation medium, in the presence of a carbon source, to provide a biomass material;
b. separating the biomass material in a first separation step, to provide a concentrated biomass material and a first liquid fraction;
c. performing an inactivation treatment on the concentrated biomass material;
d. optionally, homogenising the concentrated biomass material;
in which steps c. and d. can take place in any order,
wherein said process comprises the additional steps of:
e. treating the concentrated biomass material with an antioxidant after step b; and
f. adjusting the pH of the concentrated biomass material to a pH of 7.0 or below, after step b.

An oxidation-stabilised biomass material, in dry powder or pellet form is also provided, comprising by dry weight:
60-75% protein; preferably 65-72% protein;
1-10%, preferably 7-9% fatty acids;
0.01-2% antioxidant
wherein—when the biomass material is dissolved in water at a concentration of between 100 and 400 g/L—the resulting solution has a pH below 7.0.

Furthermore, an oxidation-stabilised biomass material, said oxidation-stabilised biomass material having a stability against oxidation of at least twice, preferably at least 3 times, that of the same biomass material which has not been subjected to stabilisation; wherein said stabilisation comprises the steps of:
treating the biomass material with an antioxidant; and
adjusting the pH of the biomass material to a pH of 7.0 or below.

Stabilisation treatment can be done in the existing downstream production but can also be implemented in other process line of single cell protein from a fermentation cultured with methanotrophic bacteria.

This, and other advantages of the invention, are set out in the following patent claims, figures and examples.

### LEGENDS TO THE FIGURES

FIG. 1 illustrates various routes of production of biomass, according to the invention.

### DETAILED DISCLOSURE

Throughout this text, the abbreviation “DM” refers to “Dry Matter”.

### SPECIFIC EMBODIMENTS

A process is thus provided for producing a biomass material which is stabilised against oxidation. Improved stability against oxidation is determined using a Oxidation stability tester, such as the ML Oxipres™ from Mikrolab Aarhus A/S.

The first step in the process is (a.) fermenting at least one methanotroph in a fermentation medium, in the presence of a carbon source, to provide a biomass material.

The biomass material is a single-cell protein (SCP) product. It comprises a majority of protein (ca. 60%), and lesser amounts of RNA and DNA. When isolated from the fermentation step, the biomass material is an aqueous suspension. In this aqueous suspension, the majority of the solid component is cellular material from the methanotroph. Other components (e.g., proteins, nucleic acids, polysaccharides, lipids or other small molecules) may be dissolved or suspended in the aqueous phase.

At least one of the microorganisms used in the fermentation step is a methanotroph, more preferably Methylococcus capsulatus. Therefore, the biomass is suitably Methylococcus capsulatus biomass.

The term “Methylococcus capsulatus” or “M. capsulatus”, as used herein, can mean any strain of bacteria belonging to the M. capsulatus species. The strain may be either naturally occurring or developed in a laboratory, such as a genetically modified strain. The term “naturally occurring” means that the strain has not been genetically modified using genetic engineering techniques. However, it may contain natural modifications or alterations in its genetic material compared to a reference strain, such as alterations that occur randomly during replication. Preferably, the strain is naturally occurring. Also preferably, the strain is M. capsulatus (Bath), more preferably the M. capsulatus (Bath) identified under NCIMB 11132. However, it may also be M. capsulatus (Texas) or M. capsulatus (Aberdeen) or a different M. capsulatus strain which is currently known or will be discovered or characterized in the future.

The methanotrophic bacteria may be provided in a co-fermentation together with one or more heterotrophic bacteria. The following heterotrophic bacteria may be particularly useful to co-ferment with M. capsulatus; Ralstonia sp.; Bacillus brevis; Brevibacillus agri; Alcaligenes acidovorans; Aneurinibacillus danicus and Bacillus firmus. Suitable yeasts may be selected from species of Saccharomyces and/or Candida. The preferred heterotrophic bacteria are chosen from Alcaligenes acidovorans (NCIMB 13287), Aneurinibacillus danicus (NCIMB 13288) and Bacillus firmus (NCIMB 13289) and combinations thereof. The methanotrophic bacteria and/or the heterotrophic bacteria may be genetically modified. In a co-fermentation, M. capsulatus make up between 90-98%.

In the fermentation step, the carbon source is converted by the microorganism(s) to biomass material. Suitably, the carbon source comprises methane, and is e.g., natural gas, syngas or biogas. During the fermentation step, the carbon source is dissolved in the fermentation medium. Fermentation suitably takes place in a U-loop reactor, as described in WO 2010/069313, hereby incorporated by reference. A suitable fermentation medium is described in e.g. WO 2018/158322 hereby incorporated by reference. The fermentation step has a relatively low Dry Matter content, e.g. below 5%.

Further details of the fermentation process are described in WO 2020/245197 and WO 2020/249670, which are hereby incorporated by reference.

The second step in the process is (b) separating the biomass material in a first separation step, to provide a concentrated biomass material and a first liquid fraction. The first separation step suitably comprises or consists of a centrifugation step, a membrane filtration step, or combination thereof, preferably wherein the first separation step comprises or consists of a centrifugation step. Concentration of the biomass takes place to provide a concentrated biomass material with a Dry Matter (DM) content of between 5-25%, preferably 10-20%.

When the first separation step is a centrifugation step, this provides a first liquid fraction in the form of a supernatant. When the first separation step is a membrane filtration step, this provides a first fraction in the form of a filtration permeate.

The process further comprises a step of (c) performing an inactivation treatment on the concentrated biomass material. This is because all living host organisms need to be killed before a final product is achieved. The inactivation treatment may comprise one or more of Ultra High Temperature (UHT) treatment, treatment with UV radiation or sterile filtration, and is preferably Ultra High Temperature (UHT) treatment. Preferably, UHT treatment takes place at a temperature of at least 120° C., preferably between 12° and 135° C. Suitably, UHT treatment is carried out for between 5 and 60 minutes.

To improve the uniformity of the concentrated biomass material the process may include an optional step of (d) homogenising the concentrated biomass material. Homogenisation typically takes place in a homogeniser vessel, at pressures between 5 and 900 bar. During homogenisation, large particles in the concentrated biomass material are broken down, to give a more uniform distribution.

The steps of inactivation and homogenisation (steps c. and d.) can take place in any order. Preferably, step c. is carried out before step d.

At this point in the process, the concentrated biomass material has a pH which is typically around pH 6.8-7.0.

According to the invention, the process comprises the additional steps of:
e. treating the concentrated biomass material with at least one antioxidant after step b; and
f. adjusting the pH of the concentrated biomass material to a pH of 7.0 or below, after step b.

As noted, both of these steps take place after step b., i.e., the separation step. Treatment with antioxidant and adjusting pH act in a synergistic manner, so as to improve the resistance of the biomass material to oxidation. In other words, it is not sufficient to only add antioxidant, or pH regulator, alone.

In one aspect, steps e. and f. are performed simultaneously. In another aspect, step f. is performed before step e. Steps e. and f. may be performed after step d, alternatively steps e. and f. may be performed before step d. Overall, the preferred process comprises steps a-f in order.

The steps of:
e. treating the concentrated biomass material with at least one antioxidant after step b; and
f. adjusting the pH of the concentrated biomass material to a pH of 7.0 or below, after step b.

may be carried out in the same vessel, suitably with agitation for at least 30 minutes. The temperature of the biomass material in this vessel can be as obtained from a preceding step (e.g. 20-40° C.) or the biomass material in this vessel can be actively cooled to 7-10° C.

In a further aspect, both step c. and step d. are carried out, and step c. is performed before step d.

The process may further comprise a step of evaporation of water, between step b. and step c, between step b. and step d, and/or between steps c. and d. Such an evaporation step typically results in a DM between 20-40%, preferably 25-35%.

Step f. is suitably carried out by adding an acid or a buffer to the concentrated biomass material, so as to arrive at the required pH. Measurement of pH of a biomass material is within the remit of the skilled person. Preferably in step f., the pH of the biomass material is adjusted to a pH of 6.5 or below, such as 6.0 or below, 5.8 or below, preferably 5.5 or below. pH adjustment may be carried out by addition of an inorganic acid or an organic acid, preferably an organic acid. In one aspect, pH adjustment is performed by addition of a C₁-C₅ organic acid, such as e.g. citric acid or propionic acid, preferably citric acid. Citric acid is preferred, due to its polyacid functionality, and the possibility of creating stable buffered solutions. Suitably—in step f.—the pH of the biomass material is adjusted to a pH of 4.0 or above, preferably 4.5 or above.

The present invention may use a range of antioxidants, such as ascorbic acid, ascorbyl stearate, tocopherols, rosemary extract, propyl gallate, quinones such as tert-butylhydroquinone (TBHQ), butylated hydroxyanisole (BHA), butylated hydroxytoluene (BHT), β-carotene, beta-apo-8′-carotenal, carotenoic acid, ethyl ester, beta-apo-8′-, citric acid, isopropyl citrates, thiodipropionic acid, dilauryl thiodipropionate, and stearyl citrate. A mixture of two or more of such antioxidants are also possible. Preferred antioxidant are ascorbic acid, ascorbyl stearate, tocopherols, rosemary extract, propyl gallate, quinones such as tert-butylhydroquinone (TBHQ), butylated hydroxyanisole (BHA), butylated hydroxytoluene (BHT), β-carotene, beta-apo-8′-carotenal, carotenoic acid, ethyl ester, beta-apo-8′-, thiodipropionic acid and dilauryl thiodipropionate. Tocopherol-containing antioxidants are most preferred, as are antioxidants from natural sources.

In one aspect, the process further comprising a final step of g. drying the biomass material to provide a powdered biomass material. Suitably, this drying step is spray-drying. The process may further comprise a step of h. pelletising the powdered biomass material to provide pelletised biomass material.

As noted above, the process of the invention provides an oxidation-stabilised biomass material. Preferably, the oxidation-stabilised biomass material has a stability against oxidation which is at least twice, preferably at least 3 times, that of the same biomass material which has not been subject to steps e. and f.

An oxidation-stabilised biomass material, in dry powder or pellet form may be produced by the process of the present invention. This material comprises by dry weight:
60-75%, preferably 65-72% protein;
1-10%, preferably 7-9% fatty acids;
0.01-2% antioxidant
wherein—when the biomass material is dissolved in water at a concentration of between 100 and 400 g/L—the resulting solution has a pH below 7.0.

An oxidation-stabilised biomass material is also provided, said oxidation-stabilised biomass material having a stability against oxidation of at least twice, preferably at least 3 times, that of the same biomass material which has not been subjected to stabilisation; wherein said stabilisation comprises the steps of:
treating the biomass material with an antioxidant; and
adjusting the pH of the biomass material to a pH of 7.0 or below.

The process and oxidation-stabilised biomass material are useful in aquatic environments (i.e. as fish feed), where the problems of oxidation are highly relevant. The invention thus provides an aquatic feed product, comprising the oxidation-stabilised biomass material as described herein, preferably in 10-30% DM.

### DETAILED DESCRIPTION

The M. capsulatus biomass can be prepared by the method described in Larsen and Jørgensen, Appl. Microbiol. Biotechnol., 1996. M. capsulatus, e.g., M. capsulatus Bath (NCIMB 11132), is grown in a bioreactor in a suitable medium with methane as the carbon source.

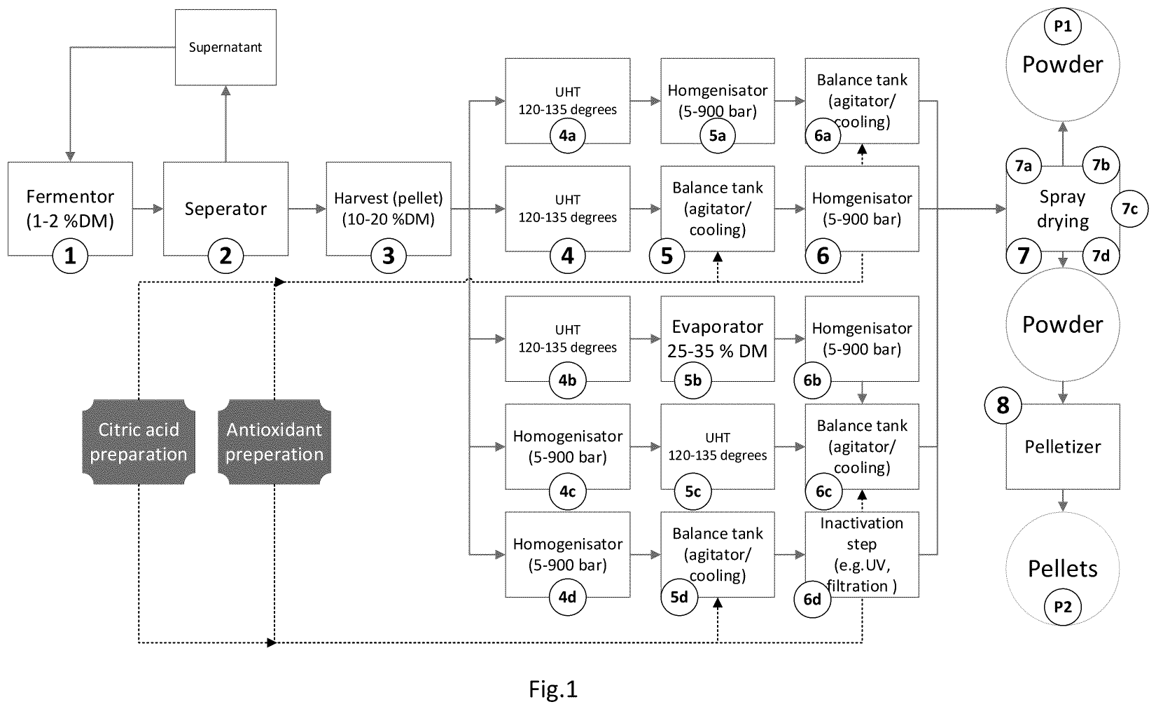

The downstream production process for biomass derived from a fermentation based on the cultivation of a methanotrophic strain is shown in FIG. 1. This downstream production concentrates the biomass from 1-2% DM to about 10-20% DM see FIG. 1 step 1 to 3. The product is then heat-treated at 120-135° C. (step 4) and then homogenized at 6-900 bar (step 5). The product can then be spray-dried (step 7) and pelleted (step 8), if desired.

This invention implements an additional treatment step see FIG. 1, where the biomass after concentration (step 3) is treated with antioxidant and the pH is changed/controlled somewhere in the already existing processes. There are 5 possible production pathways for the treatment with antioxidant and pH. All 5 possibilities are illustrated in FIG. 1. The most often-used downstream process is from step 1 to 8, where the treatment can be done in step 6 in the holding tank before spray drying.

The pH adjustment is typically performed before adding antioxidant. The pH can be adjusted with acid, where citric acid is preferable, but propionic acid can be an alternative. The pH is adjusted to below 7.0. preferably below 6.0. The addition of antioxidant: weigh the antioxidant in the amounts 0.05-3 g/I and add it after the pH adjustment. Agitation of the tank used for the treatment is usually necessary. Cooling is optional.

The invention provides single cell protein (SCP) originating from a fermentation cultured with methanotrophic bacteria against oxidation when used for e.g., fish feed formulations.

### DESCRIPTION OF THE DRAWINGS

FIG. 1 describes the five production pathways available in this invention.

1st process line includes:
1. Fermentor with 1-2% DM
2. Separator to concentrate the biomass to 10-20% DM
3. The concentrated biomass
4a. UHT treatment at 120-135° C.
5a. The biomass is homogenized at 5-900 bar
6a. Balance tank where pH regulator and antioxidant are added to treat the biomass (adjust pH to below 7.0 and add antioxidant in the range of 0.05-3 g/l)
7. The biomass is spray dried-Powder product P1 is produced
8 Powder is pelletized-Pellet product P2 is produced

Second process line includes:
1. Fermentor with 1-2% DM
2. Separator to concentrate the biomass to 10-20% DM
3. The concentrated biomass
4. UHT treatment at 120-135° C.
5. Balance tank where pH regulator and antioxidant are added to treat the biomass (adjust pH below 7.0 and add antioxidant in the range of 0.05-3 g/l)
6. The biomass is homogenized at 5-900 bar
7. The biomass is spray dried-Powder product P1 is produced
8. Powder is pelletized-Pellet product P2 is produced

Third process line includes:
1. Fermentor with 1-2% DM
2. Separator to concentrate the biomass to 10-20% DM
3. The concentrated biomass
4b. UHT treatment at 120-135° C.
5b. Evaporation of biomass to a concentration of 25-35% DM
6b. The biomass is homogenized at 5-900 bar
6c. Balance tank where pH regulator and antioxidant are added to treat the biomass (adjust pH to below 7.0 and add antioxidant in the range of 0.05-3 g/l)
7. The biomass is spray dried-Powder product P1 is produced
8 Powder is pelletized-Pellet product P2 is produced

Fourth process line includes:
1. Fermentor with 1-2% DM
2. Separator to concentrate the biomass to 10-20% DM
3. The concentrated biomass
4c. The biomass is homogenized at 5-900 bar
5c. UHT treatment at 120-135° C.
6c. Balance tank where pH regulator and antioxidant are added to treat the biomass (adjust pH to below 7.0 and add antioxidant in the range of 0.05-3 g/l)
7. The biomass is spray dried-Powder product P1 is produced
8 Powder is pelletized-Pellet product P2 is produced

Fifth process line includes:
1. Fermentor with 1-2% DM
2. Separator to concentrate the biomass to 10-20% DM
3. The concentrated biomass
4d. The biomass is homogenized at 5-900 bar
5d. Balance tank where pH regulator and antioxidant are added to treat the biomass (adjust pH to below 7.0 and add antioxidant in the range of 0.05-3 g/l)
6d. The biomass is inactivated e.g by UV radiation or filtration
7. The biomass is spray dried-Powder product P1 is produced
8 Powder is pelletized-Pellet product P2 is produced

Illustrative procedure for determining oxidation stability using ML Oxipres™.

### Procedure

1. Choose a suitable oxidation temperature (e.g. 80° C.). Turn on the heater 20 minutes before the start of the experiment.
2. Weigh a suitable amount of sample in each glass vessel. (An amount containing 3-5 g fat will often give a reasonable result). Place the glass cover on top of the vessel.
3. Place the glass vessel in the pressure vessel (bomb).
4. Make sure that the O-ring and the groove are clean. Mount the top of the pressure vessel and tighten the closure by hand.
5. Connect the pressure vessel(s) to the filling station.
6. a. Close the valve on the filling station.
b. Close the regulator outlet valve.
c. Open the main cylinder valve (slowly).
d. Adjust the outlet pressure at the regulator to 5 bar (70 psig).
e. Open the pressure vessel valve(s).
7. a. Open the regulator outlet valve.
b. Turn the filling station valve to FILL
c. To flush the pressure vessel for atmospheric air turn the valve to VENT.
d. To repeat flushing repeat b and c. (Flushing 3 times will bring the nitrogen content below 1%).
8. Now fill the pressure vessel with oxygen (FILL). Using the cylinder pressure control to reach 5 bar (70 psig) or lower. Read the pressure vessel pressure at the displays on the control unit.
9. Close the filling station valve (OFF) and then close the pressure vessel valves.
10. Watch the pressure for a few minutes to see if the pressure vessels have been tightened properly.
11. If tight turn the filling station valve to (VENT) and disconnect the filling tubes from the pressure vessels.
12. Place the pressure vessels in the block heater and start the test.
13. Remember to shut off the cylinder main valve.
14. After the test, the valve is opened. To reduce possible smell the venting can be done through a odour trap (charcoal filter) or in a fume cupboard.

### Evaluation of Results

As a result of the consumption of oxygen the pressure in the bombs will drop. (At the beginning the pressure will rise due to the heating). The signals from the pressure transducers are shown on the displays. The signals are recorded as a function of time.

The samples are characterized by the induction period. The induction period (IP, in hours) can be found by plotting the signal as a function of time, drawing tangents T1 and T2 to the curve. The time from START to the intersection is the IP. START is when the pressure vessel is placed in the block heater. The induction period will then be the time elapsed between placing the pressure vessel in the block heater and the break-point at a given temperature (e.g. 80° C.).

### Experimental Results

At the beginning of the research, the focus was on peroxide (PV) and thiobarbituric acid (TBA) as parameters for oxidation. Later, Oxipres ie. oxidation stability was used by default, due to improved reliability.

The first test (Sample A, without pH regulator or antioxidant) showed a reference stability of 17.5 hours (i.e. low stability). Addition of antioxidant alone did not provide a significant increase in stability (Sample B).

When the product was treated with different pH regulators and antioxidants in combination, (Samples C-F), up to 75 hours of stability were observed. The procedure was tested in a larger scale (Sample E) where a stability of 61.3 hours was observed. This product was non-homogenized which indicates that the treatment can take place in different types of biomass product and maintain the oxidative stability. In Sample F, another antioxidant from Vitablend was tested successfully and the treatment is therefore not dependent on one brand. Samples that absorb oxygen quickly have a low resistance to oxidation (corresponding to a low time in the Oxipres test).

TABLE 1
oxidative stability of various biomass materials, after various
treatments. The Oxipres value (in hours) corresponds to the
Induction Period determined in the Oxipres method, above

| Sample | Antioxidant | | | Oxipres |
|---|---|---|---|---|
| Name | treatment | pH regulator | pH | (hours) |
| A (ref) | None | None | 6.8 | 17.15 |
| B (ref) | Naturox IP2 | None | 7.5 | 19.5 |
| C | Naturox IP2 | Citric acid | 5 | 75.75 |
| D | Naturox IP2 | Citric acid | 4.6 | 80.2 |
| E | Naturox IP2 | Citric acid | 5 | 61.3 |
| F | Vitablend | Citric acid | 5 | 70.6 |
| G (ref) | None | Citric acid | 7.5 | 18.75 |
| H (ref) | None | Citric acid | 5.07 | 19.6 |
| I (ref) | None | Sulfuric acid | 5.02 | 17.9 |
| | | and citric | | |
| | | acid | | |
| K | None | Propionic | 5.07 | 14.9 |
| | | acid and | | |
| | | citric acid | | |
| L (ref) | None | None | 6.9 | 16.23 |
| M | Vitablend | Citric acid | 5.3 | 69.4 |
| N (ref) | Vitablend | None | 7.1 | 18.53 |
| O (ref) | None | Citric acid | 4.8 | 19.55 |

The following list of numbered aspects is provided:

Aspect 1. A process for producing a biomass material which is stabilised against oxidation, said process comprising the steps of:
a. fermenting at least one methanotroph in a fermentation medium, in the presence of a carbon source, to provide a biomass material;
b. separating the biomass material in a first separation step, to provide a concentrated biomass material and a first liquid fraction;
c. performing an inactivation treatment on the concentrated biomass material;
d. optionally, homogenising the concentrated biomass material;
in which steps c. and d. can take place in any order,
wherein said process comprises the additional steps of:
e. treating the concentrated biomass material with at least one antioxidant after step b; and
f. adjusting the pH of the concentrated biomass material to a pH of 7.0 or below, after step b.

Aspect 2. The process according to aspect 1, wherein steps e. and f. are performed simultaneously.

Aspect 3. The process according to aspect 1, wherein step f. is performed after step e.

Aspect 4. The process according to any one of the preceding aspects, wherein steps e. and f. are performed after step d, alternatively wherein steps e. and f. are performed before step d.

Aspect 5. The process according to any one of the preceding aspects, wherein the first separation step comprises a centrifugation step, a membrane filtration step, or combination thereof, preferably wherein the first separation step comprises or consists of a centrifugation step.

Aspect 6. The process according to any one of the preceding aspects, wherein the inactivation treatment is an ultrahigh temperature (UHT) treatment, preferably at a temperature of at least 120° C.

Aspect 7. The process according to any one of the preceding aspects wherein step c. is performed before step d.

Aspect 8. The process according to any one of the preceding aspects, wherein—in step f.—the pH of the biomass material is adjusted to a pH of 6.5 or below, such as 6.0 or below, 5.8 or below, preferably 5.5 or below.

Aspect 9. The process according to any one of the preceding aspects, wherein—in step f.—the pH of the biomass material is adjusted to a pH of 4.0 or above, preferably 4.5 or above.

Aspect 10. The process according to any one of the preceding aspects, wherein pH adjustment is performed by addition of a C₁-C₅ organic acid, such as e.g. citric acid or propionic acid, preferably citric acid.

Aspect 11. The process according to any one of the preceding aspects, wherein the antioxidant is selected from the group consisting of ascorbic acid, ascorbyl stearate, tocopherols, rosemary extract, propyl gallate, quinones such as tert-butylhydroquinone (TBHQ), butylated hydroxyanisole (BHA), butylated hydroxytoluene (BHT), β-carotene, beta-apo-8′-carotenal, carotenoic acid, ethyl ester, beta-apo-8′-, citric acid, isopropyl citrates, thiodipropionic acid, dilauryl thiodipropionate, stearyl citrate and mixtures of two or more of such antioxidants.

Aspect 12. The process according to any one of the preceding aspects, wherein the process further comprises a step of evaporation of water, between step b. and step c, between step b. and step d, and/or between steps c. and d.

Aspect 13. The process according to any one of the preceding aspects, further comprising a final step of g. drying the biomass material to provide a powdered biomass material.

Aspect 14. The process according to aspect 13, further comprising a step of h. pelletising the powdered biomass material to provide pelletised biomass material.

Aspect 15. The process according to any one of the preceding aspects, wherein at least one of the methanotrophs is Methylococcus capsulatus.

Aspect 16. The process according to any one of the preceding aspects, wherein the biomass is Methylococcus capsulatus biomass.

Aspect 17. The process according to any one of the preceding aspects, wherein the fermentation step (a) comprises fermenting a mixture of a methanotrophic bacteria and one or more heterotrophic bacteria.

Aspect 18. The process according to aspect 17, wherein the heterotrophic bacteria is selected from the group consisting of Ralstonia sp.; Bacillus brevis; Brevibacillus agri; Alcaligenes acidovorans; Aneurinibacillus danicus and Bacillus firmus

Aspect 19. The process according to any one of the preceding aspects, wherein the carbon source comprises methane, and is e.g., natural gas or biogas.

Aspect 20. The process according to any one of the preceding aspects, wherein steps e. and f. provide an oxidation-stabilised biomass material having a stability against oxidation which is at least twice, preferably at least 3 times, that of the same biomass material which has not been subject to steps e. and f.

Aspect 21. The process according to any one of the preceding aspects, further comprising a step of recycling the first liquid fraction from the separation step (b) to the fermentation step (a).

Aspect 22. An oxidation-stabilised biomass material, obtained from the fermentation of at least one methanotroph, in dry powder or pellet form, comprising by dry weight:
60-75%, preferably 65-72% protein;
1-10%, preferably 7-9% fatty acids;
0.01-2% antioxidant
wherein—when the biomass material is dissolved in water at a concentration of between 100 and 400 g/L—the resulting solution has a pH below 7.0.

Aspect 23. An oxidation-stabilised biomass material, obtained from the fermentation of at least one methanotroph, said oxidation-stabilised biomass material having a stability against oxidation of at least twice, preferably at least 3 times, that of the same biomass material which has not been subjected to stabilisation; wherein said stabilisation comprises the steps of:
treating the biomass material with an antioxidant; and
adjusting the pH of the biomass material to a pH of 7.0 or below.

Aspect 24. An aquatic feed product, comprising the oxidation-stabilised biomass material of any one of aspects 22-23, preferably in 10-30% DM.

### LIST OF REFERENCES

J. Larsen & L. Jørgensen Applied Microbiology and Biotechnology volume 45, pages 137-140 (1996)

1. A process for producing a biomass material which is stabilised against oxidation, said process comprising the steps of:
a. fermenting at least one methanotroph in a fermentation medium, in the presence of a carbon source, to provide a biomass material;
b. separating the biomass material in a first separation step, to provide a concentrated biomass material and a first liquid fraction;
c. performing an inactivation treatment on the concentrated biomass material;
d. optionally, homogenising the concentrated biomass material;
in which steps c. and d. can take place in any order,
wherein said process comprises the additional steps of:
e. treating the concentrated biomass material with at least one antioxidant after step b; and
f. adjusting the pH of the concentrated biomass material to a pH of 7.0 or below, after step b.

2. The process according to claim 1, wherein
steps e. and f. are performed simultaneously,
step f. is performed after step e.
step c. is performed before step d.
steps e. and f. are performed after step d, or
wherein steps e. and f. are performed before step d.

3. The process according to any one of the preceding claims, wherein the first separation step comprises a centrifugation step, a membrane filtration step, or combination thereof, preferably wherein the first separation step comprises or consists of a centrifugation step.

4. The process according to any one of the preceding claims, wherein the inactivation treatment is an ultrahigh temperature (UHT) treatment, preferably at a temperature of at least 120° C.

5. The process according to any one of the preceding claims, wherein—in step f.—the pH of the biomass material is adjusted to a pH of 6.5 or below, such as 6.0 or below, 5.8 or below, preferably 5.5 or below and/or wherein—in step f.—the pH of the biomass material is adjusted to a pH of 4.0 or above, preferably 4.5 or above.

6. The process according to any one of the preceding claims, wherein pH adjustment is performed by addition of a C₁-C₅ organic acid, such as e.g. citric acid or propionic acid, preferably citric acid.

7. The process according to any one of the preceding claims, wherein the antioxidant is selected from the group consisting of ascorbic acid, ascorbyl stearate, tocopherols, rosemary extract, propyl gallate, quinones such as tert-butylhydroquinone (TBHQ), butylated hydroxyanisole (BHA), butylated hydroxytoluene (BHT), β-carotene, beta-apo-8′-carotenal, carotenoic acid, ethyl ester, beta-apo-8′-, citric acid, isopropyl citrates, thiodipropionic acid, dilauryl thiodipropionate, stearyl citrate and mixtures of two or more of such antioxidants.

8. The process according to any one of the preceding claims, further comprising a final step of g. drying the biomass material to provide a powdered biomass material, optionally followed by a step of h. pelletising the powdered biomass material to provide pelletised biomass material.

9. The process according to any one of the preceding claims, wherein at least one of the methanotrophs is Methylococcus capsulatus.

10. The process according to any one of the preceding claims, wherein the fermentation step (a) comprises fermenting a mixture of a methanotrophic bacteria and one or more heterotrophic bacteria, preferably wherein the heterotrophic bacteria is selected from the group consisting of Ralstonia sp.; Bacillus brevis; Brevibacillus agri; Alcaligenes acidovorans; Aneurinibacillus danicus and Bacillus firmus

11. The process according to any one of the preceding claims, wherein steps e. and f. provide an oxidation-stabilised biomass material having a stability against oxidation which is at least twice, preferably at least 3 times, that of the same biomass material which has not been subject to steps e. and f.

12. The process according to any one of the preceding claims, further comprising a step of recycling the first liquid fraction from the separation step (b) to the fermentation step (a).

13. An oxidation-stabilised biomass material obtained from the fermentation of at least one methanotroph, in dry powder or pellet form, comprising by dry weight:
60-75%, preferably 65-72% protein;
1-10%, preferably 7-9% fatty acids;
0.01-2% antioxidant
wherein—when the biomass material is dissolved in water at a concentration of between 100 and 400 g/L—the resulting solution has a pH below 7.0.

14. An oxidation-stabilised biomass material obtained from the fermentation of at least one methanotroph, said oxidation-stabilised biomass material having a stability against oxidation of at least twice, preferably at least 3 times, that of the same biomass material which has not been subjected to stabilisation; wherein said stabilisation comprises the steps of:
treating the biomass material with an antioxidant; and
adjusting the pH of the biomass material to a pH of 7.0 or below.

15. An aquatic feed product, comprising the oxidation-stabilised biomass material of any one of claims 13-14, preferably in 10-30% DM.
